(19) United States
(12) Patent Application Publication
(10) Pub. No.: US 2021/0327291 A1
(43) Pub. Date: Oct. 21, 2021

(54) System and Method for Evaluating Reading Comprehension

(71) Applicant: Drexel University, Philadelphia, PA (US)

(72) Inventors: Lori Severino, Lincoln University, PA (US); Mary Jane Tecce DeCarlo, Oreland, PA (US); Meltem Izzetoglu, Drexel Hill, PA (US)

(73) Assignee: Drexel University, Philadelphia, PA (US)

(21) Appl. No.: 17/324,149

(22) Filed: May 19, 2021

Related U.S. Application Data
(63) Continuation-in-part of application No. 14/884,802, filed on Oct. 16, 2015, now abandoned.
(60) Provisional application No. 62/065,139, filed on Oct. 17, 2014.

(51) Int. Cl. G09B 7/02; A61B 5/00; G09B 17/00

(52) U.S. Cl. CPC G09B 7/02; A61B 5/0075; G09B 17/003; A61B 5/6803; A61B 5/0082

(57) ABSTRACT

A method for evaluating reading comprehension is provided. The method includes the steps of providing at least one printed passage of text; providing a test subject, the test subject wearing a device for measuring brain frontal lobe usage; requiring the test subject to read the printed passage; providing a question based on the printed passage for the test subject to answer; and determining whether the device measures brain frontal lobe usage. A system for performing the method is also provided.

### CROSS-REFERENCE TO RELATED APPLICATION

The present application is a Continuation-in-Part of U.S. patent application Ser. No. 14/884,802, filed on Oct. 16, 2015, which claims priority from U.S. Provisional Patent Application Ser. No. 62/065,139, filed on Oct. 17, 2014, both of which are incorporated by reference herein in their entireties.

### BACKGROUND OF THE INVENTION

### Field of the Invention

The present invention relates to a system and method for evaluating reading comprehension in students, and, in particular, to a system and method for validating text dependent questions of reading passages during validity and reliability stages of test development as well as validating the types of answers to provide teachers with student information.

### Description of the Related Art

Some current methods of instruction require a teacher to test the student one-on-one. Such methods do not allow for data collection and coding of incorrect answers to draw conclusions about students' areas of need. Such methods also do not allow for teacher to see growth over a short period of time and do not allow a teacher to individually test each student for 30-40 minutes every week. Few materials exist that assess reading comprehension at the secondary level and progress monitoring tools that are available do not assess reading comprehension in a way that would help teachers adapt instruction. There is a need in secondary schools for product and method that can assist teachers in this area.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a system and method for evaluating reading comprehension.

In an alternative embodiment, the present invention is a system and method for validating test questions to be used for evaluating reading comprehension.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements.

FIG. 1A is a graph of measured oxygen levels on the left and right sides of test subjects' brain frontal lobe when incorrectly answering a first text-based literal question;

FIG. 1B is a graph is a graph of measured oxygen levels on the left and right sides of test subjects' brain frontal lobe when correctly answering the first text-based literal question;

FIG. 1C is a graph is a graph of measured oxygen levels on the left and right sides of test subjects' brain frontal lobe when incorrectly answering a second text-based literal question;

FIG. 1D is a graph is a graph of measured oxygen levels on the left and right sides of test subjects' brain frontal lobe when incorrectly answering the second text-based literal question;

FIG. 2A is a graph of measured oxygen levels on the left and right sides of test subjects' brain frontal lobe when incorrectly answering a first text-based inferential question;

FIG. 2B is a graph is a graph of measured oxygen levels on the left and right sides of test subjects' brain frontal lobe when correctly answering the first text-based inferential question;

FIG. 2C is a graph is a graph of measured oxygen levels on the left and right sides of test subjects' brain frontal lobe when incorrectly answering a second text-based inferential question;

FIG. 2D is a graph is a graph of measured oxygen levels on the left and right sides of test subjects' brain frontal lobe when incorrectly answering the second text-based inferential question;

FIG. 3 is a schematic view of an fNIRS system according to an exemplary embodiment of the present invention;

FIG. 4 is a flowchart showing of a method for assessing reading comprehension according to an exemplary embodiment of the present invention;

FIGS. 5A-5D are graphs showing Maximum Oxy-Hb obtained through fNIRS vs. behavioral response time obtained through the inventive system for each subject and passage, separately;

FIG. 6A is average response times for correct and incorrect answers; and

FIG. 6B is average Oxy-Hb values for correct and incorrect answers.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term “test subject” can be used to mean a student in a classroom environment, and/or a person used to help a test developer determine whether a question on a test accurately reflects whether the question is suitable to meet the test developer's desired outcome.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Although the subject matter described herein may be described in the context of illustrative implementations to process one or more computing application features/operations for a computing application having user-interactive components the subject matter is not limited to these particular embodiments. Rather, the techniques described herein can be applied to any suitable type of user-interactive component execution management methods, systems, platforms, and/or apparatus.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Referring to the Figures in general, a system 100 for evaluating reading comprehension according to a first exemplary embodiment of the present invention is shown. System 100 is specifically developed for comprehension evaluation of students in secondary school, and can be used for other educational levels as well. System 100 contains age and grade appropriate reading passages and a plurality of related questions to each passage with multiple choice answers. Students can be tested several times during a school year with system 100 using each time a different passage and its related questions. Students can also be monitored for progress on a regular basis, such as, for example, weekly. System 100 can be downloaded and used in computers, tablets and mobile phones. System 100 has the capability to record in its log file several different pieces of information such as, for example: the date, participant information, the timings of passage reading, questions and answers, selected answers and passage reviewing times during the examination. All such information can be used for a better and more comprehensive evaluation of student's performance which is not currently possible with paper and pencil tests where only right or wrong answers and total examination time can be recorded.

In an exemplary embodiment, the 100 is reading assessment for 6th-12th grade, although those skilled in the art will recognize that system 100 can be developed for different grade levels as well. System 100 is intended to be a single piece of assessment for student data and is not meant to be the only assessment of a student's ability. System 100 is developed to assess multiple students at the same time, with test results being immediately sent to the students' teacher.

System 100 requires a test developer to develop a test with a plurality of answers including a single correct answer and a remainder of incorrect answers, or “distractors” (i.e., a multiple-choice test). The questions are developed from a particular text that a test subject will be required to read or listen to. The remainder of this disclosure, however, will be directed toward text that a test subject will be required to read.

System 100 can be used to assess one or more test subjects at the same time and can be used to provide immediate feedback on the test subjects' results. Additionally, the test subjects will be able to see graphs that explain the results in the progress that they are making. Additionally, system 100 can be used to assess validity and reliability of test questions during test development.

During test development, when developing the test questions, if, for example, four potential answers are provided, only one answer is the correct answer, with the remaining three answers being distractors. The three distractors, however, can have different levels of incorrectness. A series of tables of different types of questions and three different types of distractors (Answer Construct) for each type of question, and the goal of each distractor, is provided below. For a ten-question test based on a passage to be read, the questions include two literal questions and two inferential questions, while the remaining questions vary depending on subject matter, grade level, etc. The order of the types of questions can be shuffled for each subject based on a particular text.

| Choice for | | | |
|---|---|---|---|
| Literal | | | |
| Questions | | Answer Construct | Goal |
| Key | 3 | Correct answer | |
| Distractor 1 | 2 | Text-based literal fact, | Attract students who struggle |
| | | but with incomplete | with reading the question; |
| | | information/somewhat | students who struggle locating |
| | | related to the question | and/or retrieving info from text |
| Distractor 2 | 1 | Text-based literal fact, | Attract students who struggle |
| | | not related to question | with reading the question; |
| | | | students who struggle |
| | | | locating and/or retrieving |
| | | | info from text |
| Distractor 3 | 0 | Common background | Attract students who over rely |
| | | knowledge not in text | on prior knowledge or who do |
| | | | not read the text |
| Choice for | | | |
| Inferential | | | |
| Questions | | Answer Construct | Goal |
| Key | 3 | Correct answer | |
| Distractor 1 | 2 | Inference not supported | Attract students who are |
| | | by text | capable of inferential thinking, |
| | | | but need to attend to text clues |
| | | | (close reading) |
| Distractor 2 | 1 | Text-based literal fact | Attract students who struggle |
| | | | with reading the question or |
| | | | making inferences |
| Distractor 3 | 0 | Common background | Attract students who over rely |
| | | knowledge not in text | on prior knowledge or who do |
| | | | not read the text |

| Choice for | | | |
|---|---|---|---|
| Character/ | | | |
| Narrator | | | |
| POV (First | | | |
| Person, | | | |
| Third | | Answer | |
| Person . . . ) | | Construct | Goal |
| Key | 3 | Correct answer | |
| Distractor 1 | 2 | Incorrect answer, | Attract students who can identify |
| | | POV of different | point of view, but who not read |
| | | character/narrator | the prompt correctly |
| Distractor 2 | 1 | Reasonable answer | Attract students who understand |
| | | related to question, | the point of view, but who did not |
| | | not a text-based fact | read or comprehend text |
| Distractor 3 | 0 | Text-based fact | Attract students who may |
| | | related to character | comprehend the story, but cannot |
| | | in prompt, not | related identify point of view; |
| | | to point of view | attract students who do not read |
| | | | the prompt |

| Choice for | | | |
|---|---|---|---|
| Evidence | | | |
| for develop- | | | |
| ing POV | | Answer Construct | Goal |
| Key | 3 | Correct answer | |
| Distractor 1 | 2 | Evidence supports | Attract students who recognize |
| | | POV in general, but | point of view, but are not yet |
| | | weak evidence from | adept at identifying best evidence |
| | | unrelated part of text | |
| Distractor 2 | 1 | Evidence supports | Attract students who recognize |
| | | POV, but is not in | point of view, but who did not read |
| | | text | or comprehend text |
| Distractor 3 | 0 | Evidence from text, | Attract students who may |
| | | does not support | comprehend the story, but cannot |
| | | POV | identify point of view; attract |
| | | | students who do not read the |
| | | | prompt |

| Choice for | | |
|---|---|---|
| Vocabulary | | |
| Questions | Answer Construct | Goal |
| Key | Correct answer | |
| Distractor | Definition of a word closely | Attract students who rely |
| 1 | related to the key; synonym | on semantic cues |
| | OR alternate meaning of | |
| | vocabulary word not used | |
| | in this sentence | |
| Distractor | Definition of a word with a | Attract students who rely |
| 2 | simpler meaning | on semantic cues, but lack |
| | | sophisticated vocabulary |
| Distractor | Definition of a word that | Attract students who rely |
| 3 | would fit syntactically in | on syntax over semantics or |
| | the sentence OR literal | rely on literal word mean- |
| | interpretation of figurative | ings and do not recognize |
| | language | figurative language |

| Choice for Figurative | | | |
|---|---|---|---|
| Language Questions | | Answer Construct | Goal |
| Key | 3 | Correct answer | |
| Distractor 1 | 2 | Meaning that is either | Attract students who recognize |
| | | too strong or weak for | the figurative language and its |
| | | the phrase (i.e. | context, but fail to accurately |
| | | “raining cats and | interpret it. |
| | | dogs” means | |
| | | “drizzling”) | |
| Distractor 2 | 1 | Meaning that is | Attract students who over rely |
| | | possible in the story, | on context. |
| | | but doesn't align with | |
| | | that exact phrase. | |
| Distractor 3 | 0 | Reasonable literal | Attract students who only read |
| | | meaning of the phrase | the question or who cannot use |
| | | | context to determine meaning. |
| Choice for | | | |
| Summary or Main | | | |
| Idea | | Answer Construct | Goal |
| Key | 3 | Correct answer | |
| Distractor 1 | 2 | Main idea of one | Attract students who read portion |
| | | paragraph/portion | of text; attract students who can |
| | | | only synthesize sections of section |
| | | | of text |
| Distractor 2 | 1 | Correct author's | Attract students who over confuse |
| | | purpose: inform, | main idea with author's purpose |
| | | persuade, entertain | |
| Distractor 3 | 0 | Text-based literal fact | Attract students who can read for |
| | | | detail but not synthesize |
| | | | information |
| Choice for Part to | | | |
| the Whole | | Answer Construct | Goal |
| Key | 3 | Correct answer | |
| Distractor 1 | 2 | Correct connection, but | Attract students who can make |
| | | with incorrect evidence | connections from part to whole, |
| | | | but need to select better evidence |
| Distractor 2 | 1 | Incorrect connection | Attract students who can recall |
| | | supported with a text- | details, but fail to connect part to |
| | | based fact | the whole |
| Distractor 3 | 0 | Reasonable answer to | Attract students who understand |
| | | the question, but not | the structure element in the |
| | | related to the overall | prompt, but who did not read or |
| | | structure of this text | comprehend text |
| Choice for Best | | | |
| Evidence | | Answer Construct | Goal |
| Key | 3 | Correct answer | |
| Distractor 1 | 2 | Related fact from text | Attract students who are capable |
| | | that does not provide | identifying evidentiary facts, but |
| | | “best” evidence | not able to evaluate strongest |
| | | | choice |
| Distractor 2 | 1 | Text-based literal fact, | Attract students who can read for |
| | | not related to the | detail but can't identify evidence |
| | | question | that supports a specific conclusion |
| Distractor 3 | 0 | Supportive, evidentiary | Attract students who over rely on |
| | | fact not in text | prior knowledge or who do not |
| | | | read the text |
| Choice for Author's | | | |
| Purpose | | Answer Construct | Goal |
| Key | 3 | Correct answer | |
| Distractor 1 | 2 | Incorrect author's | Attract students who can not |
| | | purpose | distinguish between/among |
| | | | purposes |
| Distractor 2 | 1 | Correct main idea | Attract students who over confuse |
| | | | main idea with author's purpose |
| Distractor 3 | 0 | Text-based literal fact | Attract students who can read for |
| | | | detail but not synthesize |
| | | | information |
| Choice for Text | | | |
| Type or Genre | | Answer Construct | Goal |
| Key | 3 | Correct answer | |
| Distractor 1 | 2 | Incorrect answer but | Attract students who understand |
| | | text-based | the genre, but can not recall or |
| | | fact/inferences, related | infer enough information to make |
| | | to correct text type | correct selection |
| Distractor 2 | 1 | Text-based | Attract students who may |
| | | fact/inference, incorrect | comprehend the story, but |
| | | text type | cannot distinguish among text |
| | | | types; attract students who do |
| | | | not read the prompt |
| Distractor 3 | 0 | Reasonable answer | Attract students who can dis- |
| | | related to question, not | tinguish among text types, but |
| | | a text-based fact | who did not read or comprehend |
| | | | text |
| Choice for | | | |
| Relationships between | | | |
| Story Elements | | Answer Construct | Goal |
| Key | 3 | Correct answer | |
| Distractor 1 | 2 | Both facts are in the | Attract students who are capable |
| | | text, but one does | identifying evidentiary facts, but |
| | | not relate to the | not able to make causal |
| | | other | connections. |
| Distractor 2 | 1 | Text-based literal | Attract students who can read for |
| | | fact related to one of | detail but can't identify evidence |
| | | the elements but not | that supports a specific |
| | | the other | connection. |
| Distractor 3 | 0 | Supportive, | Attract students who over rely on |
| | | evidentiary facts not | prior knowledge or who do not |
| | | in text | read the text |
| Choice for Key Idea | | Answer Construct | Goal |
| Key | 3 | Correct answer | |
| Distractor 1 | 2 | Related example from | Attract students who are capable |
| | | text that does not | identifying evidentiary examples, |
| | | provide “best” support | but not able to evaluate strongest |
| | | for stated key idea | choice |
| Distractor 2 | 1 | Text-based literal | Attract students who can read for |
| | | example, not related to | detail but can't identify evidence |
| | | the question | that supports a key idea |
| Distractor 3 | 0 | Supportive, evidentiary | Attract students who over rely on |
| | | fact not in text | prior knowledge or who do not read |
| | | | the text |
| Choice for Author's | | | |
| Purpose | | Answer Construct | Goal |
| Key | 3 | Correct answer | |
| Distractor 1 | 2 | Incorrect author's | Attract students who cannot |
| | | purpose | distinguish between/among |
| | | | purposes |
| Distractor 2 | 1 | Correct main idea | Attract students who over confuse |
| | | | main idea with author's purpose |
| Distractor 3 | 0 | Text-based literal fact | Attract students who can read for |
| | | | detail but not synthesize information |
| Choice for Text | | | |
| Structure (Problem- | | | |
| solution, description, | | | |
| explanatory cause- | | | |
| effect, enumeration, | | | |
| categorization, | | | |
| sequence, | | | |
| comparison-contrast, | | | |
| narrative) | | Answer Construct | Goal |
| Key | 3 | Correct answer | |
| Distractor 1 | 2 | Incorrect answer | Attract students who understand |
| | | but text-based, | that part of structure, but cannot |
| | | correct structure | recall or infer enough information |
| | | | to make correct selection |
| Distractor 2 | 1 | Text-based fact, | Attract students who may |
| | | incorrect structure | comprehend the story, but cannot |
| | | | identify correct text structure form |
| | | | question; attract students who do |
| | | | not read the prompt |
| Distractor 3 | 0 | Reasonable answer | Attract students who understand |
| | | related to question, | the structure element in the prompt, |
| | | not a text-based | but who did not read or |
| | | fact | comprehend text |
| Choice for Text | | | |
| Features (captions, | | | |
| graphics, charts, | | | |
| diagrams, graphs) | | Answer Construct | Goal |
| Key | 3 | Correct answer | |
| Distractor 1 | 2 | Incorrect answer | Attract students who identify the |
| | | but text-based | correct feature, but cannot recall or |
| | | fact/inferences, | infer enough information to make |
| | | related to correct | correct selection |
| | | feature | |
| Distractor 2 | 1 | Text-based | Attract students who may recall the |
| | | fact/inference, | text, but cannot distinguish among |
| | | incorrect text | text features; attract students who |
| | | feature | do not read the prompt |
| Distractor 3 | 0 | Reasonable answer | Attract students who can distinguish |
| | | related to question, | among text features, but who did |
| | | not a text-based | not read or comprehend text |
| | | fact | |
| Choice for Word | | | |
| Choice and Tone | | Answer Construct | Goal |
| Key | 3 | Correct answer | |
| Distractor 1 | 2 | Words can connote stated | Attract students who understand |
| | | mood, but mood is not | how word choice can suggest |
| | | the correct one for the | mood, but who need to integrate |
| | | text | that knowledge with the story |
| | | | context |
| Distractor 2 | 1 | Words do not connote the | Attract students who recall details |
| | | stated mood, but are | from the text, but do not connect |
| | | words from the text | word choice to mood of the text |
| Distractor 3 | 0 | Words can connote stated | Attract students who over rely on |
| | | mood, those words are | prior knowledge or who do not |
| | | not from text | read the text |
| Choice for Theme | | Answer Construct | Goal |
| Key | 3 | Correct answer | |
| Distractor 1 | 2 | Theme of one part | Attract students who read portion |
| | | of the text, but not | of text; attract students who can |
| | | the text in its | only synthesize sections of section |
| | | entirety | of text |
| Distractor 2 | 1 | Correct mail idea | Attract students who over confuse |
| | | | theme with main idea |
| Distractor 3 | 0 | Text-based literal | Attract students who can read for |
| | | fact | detail but not synthesize |
| | | | information |
| Choice for | | | |
| Compare/Contrast | | Answer Construct | Goal |
| Key | 3 | Correct answer | |
| Distractor 1 | 2 | Incorrect similarity or | Attract students who can recall |
| | | difference based on | facts, but not accurately |
| | | some text-based facts | compare/contrast |
| Distractor 2 | 1 | Statement that supports | Attract students who cannot |
| | | opposite of the prompt | distinguish between compare and |
| | | | contrast |
| Distractor 3 | 0 | Similarity or difference | Attract students who over rely on |
| | | based on common | prior knowledge or who do not |
| | | background knowledge | read the text |
| | | not in text | |
| Choice for | | | |
| Compare/Contrast | | | |
| with Core Texts | | Answer Construct | Goal |
| Key | 3 | Correct answer | |
| Distractor 1 | 2 | Incorrect similarity or | Attract students who can recall |
| | | difference based on | facts, but not accurately |
| | | some text-based | compare/contrast |
| | | facts | |
| Distractor 2 | 1 | Statement that | Attract students who cannot |
| | | supports opposite of | distinguish between compare and |
| | | the prompt | contrast |
| Distractor 3 | 0 | Similarity or | Attract students who over rely on |
| | | difference based on | prior knowledge or who do not |
| | | common background | read the text |
| | | knowledge not in text | |
| Choice for Text | | | |
| Structure | | | |
| (how parts contribute | | | |
| to the whole) | | Answer Construct | Goal |
| Key | 3 | Correct answer | |
| Distractor 1 | 2 | Part that does | Attract students who can identify |
| | | not support the | the structure, but not correctly |
| | | stated whole | identify how the part supports |
| | | | the whole |
| Distractor 2 | 1 | Text-based part, | Attract students who recall text |
| | | incorrect | facts, but did not understand text |
| | | structure | structure |
| Distractor 3 | 0 | Reasonable | Attract students who understand |
| | | answer related | the structure element in the |
| | | to question, not | prompt, but who did not read or |
| | | based on text | comprehend text |
| Choice for | | | |
| Compare/Contrast | | | |
| with Outside | | | |
| Texts/Events | | Answer Construct | Goal |
| Key | 3 | Correct answer | |
| Distractor 1 | 2 | Statement that supports | Attract students who cannot |
| | | opposite of the prompt | distinguish between compare |
| | | | and contrast |
| Distractor 2 | 1 | Similarity or difference | Attract students who over rely |
| | | based on common | on prior knowledge or who do |
| | | background knowledge | not read the text |
| | | not in text | |
| Distractor 3 | 0 | Incorrect similarity or | Attract students who can recall |
| | | difference based on some | facts, but not accurately |
| | | text-based facts | compare/contrast |

Questions 1 and 2 of each passage are literal questions. Literal question answers can be found directly in the text. In functional near infrared spectroscopy (fNIRS) analysis, literal questions required more oxygenation on the left frontal lobe when students answered the question correctly. The left side of the frontal lobe has been associated with working memory. In answering literal questions, subjects need to use their working memory in order to answer the question correctly. Working memory includes holding the information for a short time in order to manipulate or otherwise do something with the information. In this case, subjects read a passage and the first two questions asked about the passage are literal questions that refer directly to the passage and what was just read (in working memory).

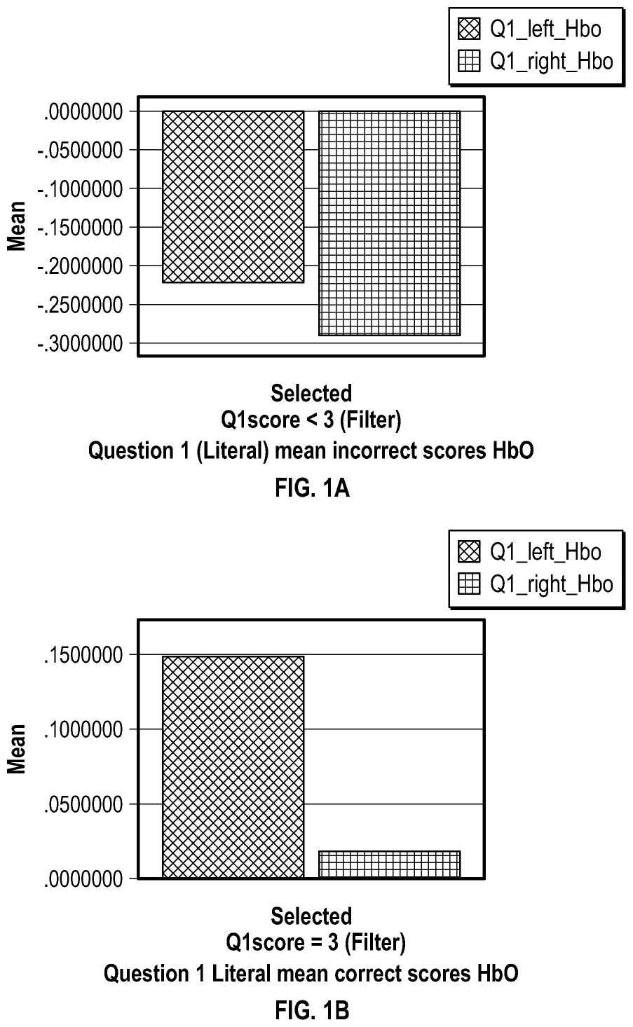

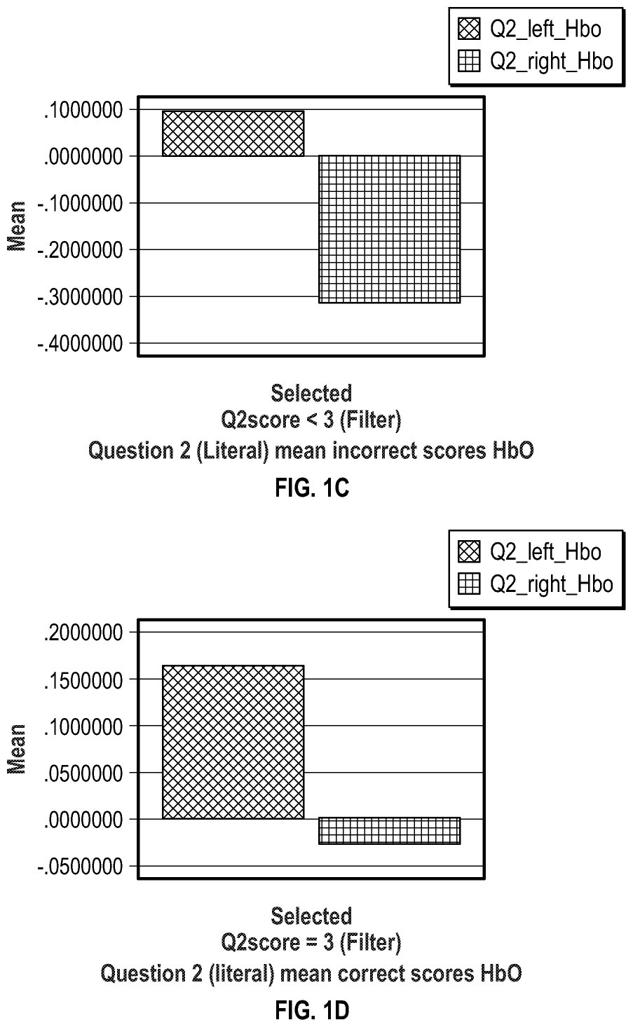

Referring to FIGS. 1A-1D, for two literal based questions, the average HbO found on the left frontal lobe=0.148448399 (Question 1) and 0.163853076 (Question 2). The negative numbers shown in FIGS. 1A, 1C, and 1D indicate no activation of that brain frontal lobe section. The high positive numbers for the left lobe shown in FIGS. 1B and 1D indicate that the left frontal lobe (working memory) was activated in arriving at the correct answer. The negative values for the right frontal lobe in FIGS. 1A, 1C, and 1D, as well as the low positive value in FIG. 1B, indicates little or no use of the right frontal lobe in answering the question either correctly or incorrectly.

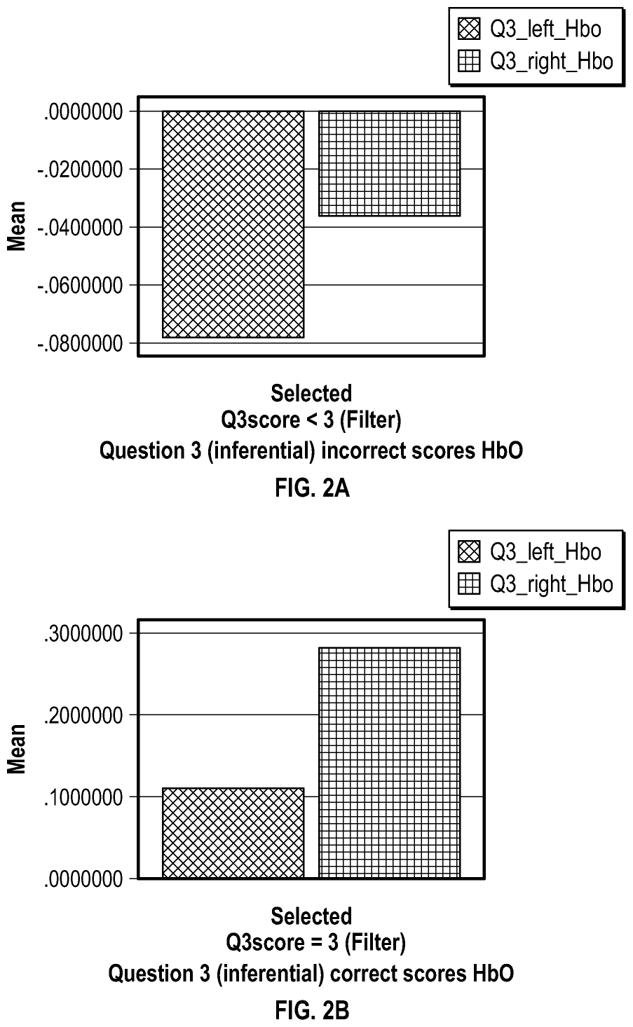

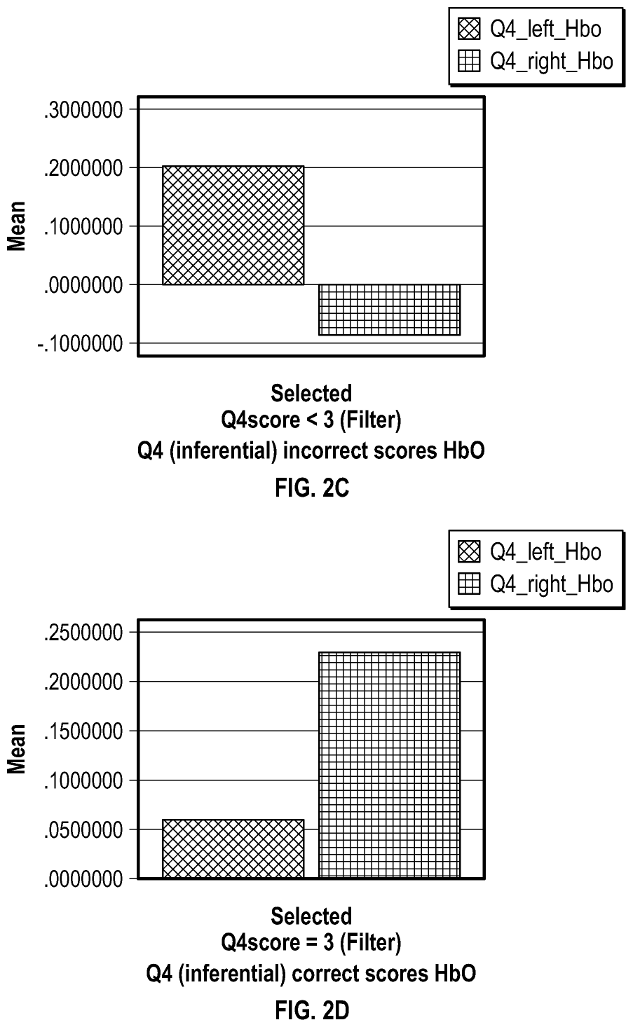

Referring now to FIGS. 2A-2D, questions 3 and 4 in each passage are inferential questions. In order for a subject to correctly answer an inferential question, the subject needs to use partly what is in the text and partly what the subject knows from experience or background knowledge. Background knowledge is likely stored in long term memory and would activate a different part of the brain than the frontal lobe. Using fNIRS, data analysis results show positive oxygenation in the right frontal lobe which is associated with attention. While activation was present on both the left and right frontal lobe, the oxygenation was higher on the right frontal lobe.

For the two exemplary inferential questions whose results are shown in FIGS. 2A-2D, the average Hb) found on the right frontal lobe=0.282245616 (Question 3) and 0.229836028 (Question 4). The negative values in FIGS. 2A and 2C indicate no brain frontal lobe activity when answering incorrectly, while the positive values in FIGS. 2B and 2D indicate both left frontal lobe activity (working memory) and right frontal lobe activity (attention) to correctly answer the questions.

In a paired T test of channel 6 & 12 of Q4 (with Channel 6 being the left frontal lobe and channel 12 the right frontal lobe) statistical significance (p=0.053) was nearly approached with n=9.

For example, a first distractor can be a text-based literal fact that is not related to the question and is designed to attract students who struggle with reading the question and students who struggle locating and/or retrieving information from the text. A second distractor is a text-based literal fact with incomplete information that is somewhat related to the question and is designed to attract students who struggle with reading the question and students who struggle locating and/or retrieving information from the text. The third distractor relates to common background knowledge not in the text, and is designed to attract students who over rely on prior knowledge or who do not read the text.

In an exemplary embodiment, when grading a test, the grading scale can be set such that the different answers have different score values. For example, the correct answer is worth 3 points, the first distractor can be worth 2 points, the second distractor can be worth 1 point, and the third distractor can be worth 0 points. With this scoring scheme, if, for example, a test has 10 questions, then the highest score would be 30 points. Subsequent testing (using different passages) may be used to determine if a test subject is doing a better job of reading and evaluating the text, but still getting incorrect answers. For example, if, during the first round of testing, the test subject got questions wrong and selected the second or third distractor in an amount of the questions, but if, during a second round of testing, the test subject, while still selecting incorrect answers, selected the first distractors in an amount of the questions, it may be able to be determined that, even though the test subject is still selecting incorrect answers, the test subject is doing a better job at reading and comprehending the text, which may correlate with a change in the test subject's brain function over time.

During test development and validation, to assist the test developer in determining whether the test subject is answering the question based on his/her recent reading of the text, fNIRS can be used. It is known that fNIRS can be used to measure brain frontal lobe usage. It is also known that the frontal lobe is, among other functions (e.g., working memory, executive functions, decision making, problem solving, attention, conflict resolution, etc.), the source of short-term memory in humans. Therefore, fNIRS can be used to determine whether or not a test subject uses his/her frontal lobe to answer a question based on a recently read passage.

By applying fNIRS hardware to a test subject to validate the test questions, if fNIRS results indicate that the test subject used his/her prefrontal cortex (where short-term memory is located) to answer the question, it can be determined that the test subject is basing his/her answer on recently read material, as desired by the test developer. The test subject would typically use brain frontal lobe to select either the correct answer or one of the first two distractors and does not use the brain frontal lobe to answer the third distractor.

Additionally, while the examples provided herein are text passages with words that comprise stories, it is within the scope of the present invention that the text can be numerals as well, requiring the test subject to perform mathematical calculations, with numerical answers as the correct answer and the distractors. For example, the multiplication text problem of 8×7 will have the correct answer of 56, a first distractor of 54 (which may indicate that the test subject tried to multiply the numbers and simply arrived at the wrong answer), a second distractor of 15 (which may indicate that the test subject added the numbers instead of multiplied the numbers), and a third distractor of 87 (which may indicate that the test subject merely put the 8 and the 7 together to form 87.

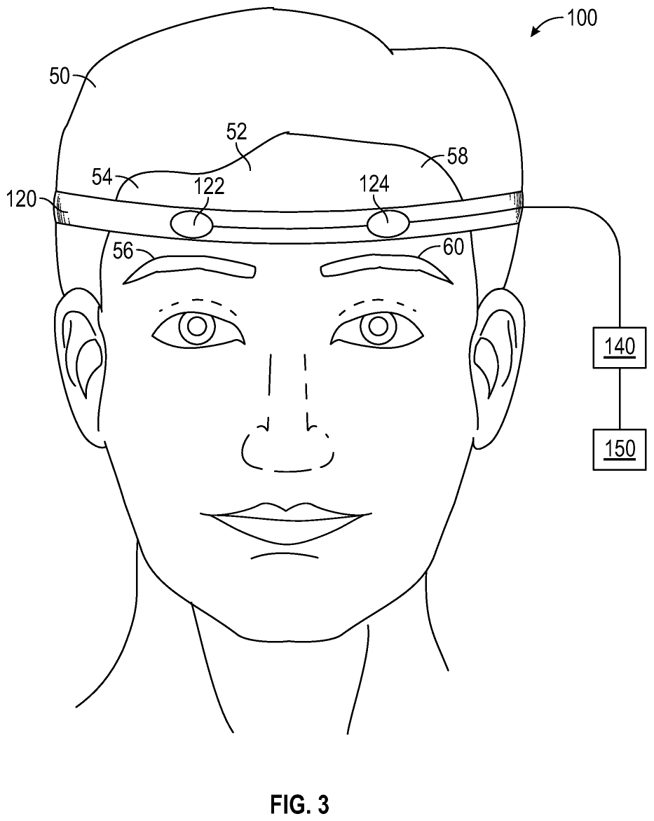

A schematic drawing of an exemplary fNIRS system 110 for use with system 100 is shown FIG. 3. The fNIRS system 110 used was a 4-channel fNIRS spectroscopy system produced by fNIR Device, LLC. The fNIRS system 110 included a head band type sensor assembly 120, data collection box 140 and a computer 150. The sensor assembly 120 is composed of two identical sensors 122, 124, each containing one light source with built in LEDs at 730 and 850 nm wavelength and 2 light detectors on each side of the light source approximately 2.5 cm away from the light source. The sensors 122, 124 were placed symmetrically on the forehead 52 of the test subject 50, one sensor 122 on the right hemisphere 54 above the right eyebrow 56 and the other sensor 124 on the left hemisphere 58 right above the left eyebrow 60, mapping the middle frontal cortex at four channel locations, where channel 1 was imaging the left most frontal area; channel 2 was on the left middle; channel 3 was on the right middle; and channel 4 was imaging the right most area on the frontal cortex. Data collection box 140 and the computer 150 are used to collect and store the data. fNIRS data is collected while students were subjected to system 100 simultaneously where time synchronization is achieved through markers.

If the fNIRS system 110 determines that the test subject used his/her brain frontal lobe to answer the question, but selected a distractor instead of the correct answer, the question can still be determined to be a question that requires brain frontal lobe usage to answer and, therefore, is a valid question based on the text. It can be noted, however, that, if many or all of the test subjects incorrectly answer the question, even if the fNIRS results indicate that the test subjects used their brain frontal lobe to answer the question, the question may need to be reworded or dropped entirely.

If, however, the fNIRS results indicate that the test subject did not use his/her brain frontal lobe to answer the question, it can be determined that the test subject may not have read the passage and that the question may not be suitable to determine the test subject's comprehension of the recently read text.

Additionally, if the test subject selected one of the distractors, the test subject can be directed to re-read the passage and answer the question again. If the answer is still wrong, but is “less” wrong than the first wrong answer (i.e., the first wrong answer was the third distractor and the second wrong answer was the second distractor), then it can be determined that the test subject appears to be making progress in comprehending the text.

An exemplary reading passage, along with a correct answer and three different types of distractors, is provided below.

A Liger's Tale
What do you get when you cross a lion with a tiger? A liger, of course! There are not a lot of ligers in the world, but one, named Hercules, made a big splash recently at Miami's Parrot Jungle Island. “It's not something you see every day,” the animal's owner, Bhagavan Antle, told New York's Daily News.
How did Hercules, who weighs 900 pounds, come to be? Three years ago [2002], his father, a lion, and his mother, a tiger, spotted each other at Antle's South Carolina animal preserve. It was love at first roar. “We have a big free-roaming area at the preserve,” Antle told the New York Post. “Sometimes lions and tigers are allowed to go out there and, lo and behold, one particular lion fell in love with one particular tiger and we had babies.” Four, to be exact: Hercules has three brothers—Vulcan, Zeus, and Sinbad.
What do ligers look like? A liger has a thick mane like that of a lion and stripes like those of a tiger. Hercules can consume 100 pounds of raw meat a day. He is able to run as fast as 50 miles per hour. At 3 years old, he's only a baby.
Does Hercules roar like a tiger or a lion? He has his dad's voice, although he swims like his mom. Like most lions, his dad doesn't enjoy the water. Hercules is special because there are no ligers in the wild. Several have been born in captivity, including one last year in a zoo in Russia. That liger's name is Zita. Ligers are rare because tigers and lions don't usually get along. “Normally the lion will kill the tiger,” Antle said.

Question:

1. Why are ligers rare?
A. Lions and tigers don't usually get along (correct answer).
B. The lion and tiger fell in love (Text-based literal fact, not related to question; Attract students who struggle with reading the question; students who struggle locating and/or retrieving info from text).
C. There are no ligers in the wild (Text-based literal fact, but with incomplete information/somewhat related to the question, Attract students who struggle with reading the question; students who struggle locating and/or retrieving info from text).
D. Ligers are unfamiliar to many people (Common background knowledge not in text, Attract students who over rely on prior knowledge or who do not read the text).

It may be desired to use original text passages and not use prior written text passages that the test subject may have had an opportunity to previously read. This will ensure that the text passage is brand new to the test subject.

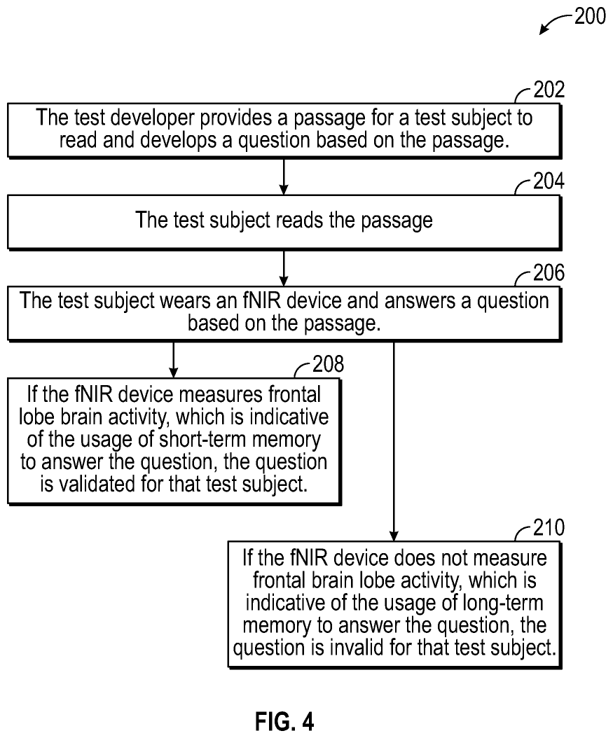

An exemplary use of the system 100 and method according to the present invention is shown in flowchart 200, shown FIG. 4. In step 202, the test developer provides a passage for a test subject to read and develops a question based on the passage. In step 204, the test subject reads the passage. In step 206, the test subject wears an fNIRS device and answers a question based on the passage. In an exemplary embodiment, only frontal lobe usage is measured.

In step 208, if the fNIRS device measures frontal lobe brain activity, which is indicative of the usage of short-term memory to answer the question, the question is validated for that test subject. In step 210, however, if the fNIRS device does not measure frontal brain lobe activity, the question is invalid for that test subject.

Steps 204-210 can be repeated for a plurality of test subjects and for a plurality of text passages. In an exemplary embodiment, the plurality of students can be at least 20 students. After the plurality of test subjects have perform steps 204-210, if a significant number, such as, for example, over 75%, of the test subjects used brain frontal lobe to answer the question, the question is validated for the test. If, however, less than the significant number of the test subjects used brain frontal lobe to answer the question, the test developer can make the decision that the question is invalid and discard the question as relates to the passage.

A plurality of questions can be developed for the passage using steps 204-210. After the test has been developed for the particular passage, steps 202-210 can be repeated, with a different passage being selected in step 202.

An exemplary use of system 100 is provided in the following example:

### Example 1

Participants and Task: 3 middle school students (age=12(mean)-males) had taken part in a preliminary study using system 100. Students performed 4 sessions using system 100 with 5 minutes to 1 hour in between sessions. In each session students were given a different passage and 10 questions to be answered related to the passage. Students and their corresponding passages in the order they have received them are given in Table 1 below.

TABLE 1
Students and the passages they had performed
in the order they had performed it

| Session | Student #10 | Student #15 | Student #20 |
|---|---|---|---|
| 1 | Phantom Tollbooth | Hatchet | Liger's Tale* |
| 2 | Liger's Tale* | Dynamic Duo | Hatchet |
| 3 | Dynamic Duo | Liger's Tale* | Dynamic Duo |
| 4 | Hatchet | Phantom Tollbooth | Front of the Bus |

*Passages where simultaneous recordings from system 100 incorporating fNIRS were collected

Results:

Behavioral Outcomes (from System 100):

Two types of analyses were performed in order to show the additional capabilities of system 100 in student performance evaluation in comparison to paper and pencil test methods. First, only the gross outcomes, such as overall testing time and correct/incorrect answers, were analyzed where it could have been accessed when paper and pencil tests were used. Then, the detailed results from system 100, such as individual question response times, number of viewing the essay during the examination, etc., were analyzed to show the efficacy of the 100 in providing valuable information in addition to the gross measurements.

Table 2 below reports on the overall timing of the test and the number of correct answers (out of 10 questions) for each passage and students as shown in Table 2. Note that if there are multiple answers given for an individual question, the last answer is taken as the answer for that question.

TABLE 2
Overall test completion time and correct answers given for each subject
and passage

| | | Correct Answers | Test Completion |
|---|---|---|---|
| Subject | Passages | Given (out of 10) | time (s) |
| #10 | Phantom Tollbooth | 5 | 370 |
| #10 | Liger's Tale* | 7 | 259 |
| #10 | Dynamic Duo | 8 | 858 |
| #10 | Hatchet | 6 | 425 |
| #15 | Hatchet | 6 | 510 |
| #15 | Dynamic Duo | 7 | 645 |
| #15 | Liger's Tale* | 7 | 292 |
| #15 | Phantom Tollbooth | 4 | 707 |
| #20 | Liger's Tale* | 7 | 381 |
| #20 | Hatchet | 7 | 257 |
| #20 | Dynamic Duo | 8 | 858 |
| #20 | Front of the Bus* | 8 | 572 |

*the passages where simultaneous recordings from system 100 and fNIRS are collected

From these overall measures, no improvement (due to practice) or deterioration (due to fatigue) is found in terms of correct answers given, although the results indicate that it appears to take more time for the students to perform the overall test in the later sessions as compared to the former ones. This increase in time in test completion is not reflected in the number of correct answers given (correlation coefficient R=0.17). Another observation here is, overall, the “tiger's Tale” passage took the least time to complete and the “Dynamic Duo” passage took the most time, which may be due to the difficulty levels of these passages. Overall, subject #20 performed the best and subject #15 performed the worst out of the three students.

Additional detailed measurements from system 100: An example use log for system 100 is given in Table 3 below. From this log, the time it took for the student to read the passage, number and timing of going back to the passage, timing of each question and the corresponding answer, response type in terms of which multiple choice is selected and if it is correct or wrong can be extracted which can provide the teacher a rich amount of information to better evaluate the student's performance.

TABLE 3
An example log for subject 15, passage “Hatchet”

| | | | | Re- | Correct |
|---|---|---|---|---|---|
| Event | Time(abs) | Time | Question | sponse | Answer |
| Started Reading | 1408541449 | 0 | | | |
| Question Start | 1408541722 | 273 | | | |
| Response | 14085411728 | 279 | 1 | 3 | 0 |
| Next Question | 1408541730 | 281 | | | |
| Response | 1408541736 | 287 | 2 | 1 | 1 |
| Response | 1408541736 | 287 | 2 | 1 | 1 |
| Next Question | 1408541738 | 289 | | | |
| Response | 1408541761 | 312 | 3 | 2 | 0 |
| Go To Essay | 1408541766 | 317 | | | |
| Question start | 1408541787 | 338 | | | |
| Response | 1408541788 | 339 | 3 | 4 | 0 |
| Response | 1408541789 | 340 | 3 | 4 | 0 |
| Next Question | 1408541790 | 341 | | | |
| Go To Essay | 1408541802 | 353 | | | |
| Question Start | 1408541809 | 360 | | | |
| Response | 1408541810 | 361 | 4 | 1 | 1 |
| Response | 1408541811 | 362 | 4 | 1 | 1 |
| Next Question | 1408541812 | 363 | | | |
| Response | 1408541837 | 388 | 5 | 2 | 0 |
| Response | 1808541837 | 388 | 5 | 2 | 0 |
| Response | 1408541838 | 389 | 5 | 2 | 0 |
| Next Question | 1408541839 | 390 | | | |
| Response | 1408541902 | 453 | 6 | 1 | 1 |
| Next Question | 1408541903 | 454 | | | |
| Response | 1408541913 | 464 | 7 | 2 | 0 |
| Response | 1408541914 | 465 | 7 | 1 | 1 |
| Next Question | 1408541922 | 473 | | | |
| Response | 1408541930 | 481 | 8 | 3 | 0 |
| Response | 1408541937 | 488 | 8 | 4 | 0 |
| Response | 1408541938 | 489 | 8 | 1 | 1 |
| Next Question | 1408541939 | 490 | | | |
| Response | 1408541952 | 503 | 9 | 3 | 0 |
| Next Question | 1408541953 | 504 | | | |
| Response | 1408541956 | 507 | 10 | 1 | 1 |
| Complete | 1408541959 | 510 | | | |

Here, as an example for additional behavioral measure analysis using system 100 logs, individual passage reading times, total number of answers given (including multiple answers for a single question), overall additional passage viewing times during the testing, the average response times for the 10 questions together with the answer types (correct answers) and overall testing time were extracted and summarized in Table 4 below.

TABLE 4
10 Question averaged values for each subject, session and passage

| | | | Pas- | | | Re- | | Over- |
|---|---|---|---|---|---|---|---|---|
| | | | sage | # of | # of | sponse | | all |
| Sub- | Ses- | | Time | An- | Go | Time | Correct | Time |
| ject | sion | Passage | (s) | swers | Essay | (s) | Answer | (s) |
| 10 | 1 | Phantom | 180 | 14 | 0 | 16.7 | 5 | 370 |
| | | Tollbooth | | | | | | |
| 10 | 2 | Liger's | 123 | 11 | 0 | 12 | 7 | 259 |
| | | Tale* | | | | | | |
| 10 | 3 | Dynamic | 181 | 12 | 1 | 19 | 8 | 858 |
| | | Duo | | | | | | |
| 10 | 4 | Hatchet | 219 | 11 | 2 | 12.2 | 6 | 425 |
| 15 | 1 | Hatchet | 273 | 19 | 1 | 19.7 | 6 | 510 |
| 15 | 2 | Dynamic | 125 | 19 | 1 | 17.8 | 7 | 645 |
| | | Duo | | | | | | |
| 15 | 3 | Liger's | 108 | 13 | 1 | 12.6 | 7 | 292 |
| | | Tale* | | | | | | |
| 15 | 4 | Phantom | 359 | 14 | 2 | 24.3 | 4 | 707 |
| | | Tollbooth | | | | | | |
| 20 | 1 | Liger's | 142 | 10 | 2 | 15.5 | 7 | 381 |
| | | Tale* | | | | | | |
| 20 | 2 | Hatchet | 26 | 10 | 0 | 20.4 | 7 | 257 |
| 20 | 3 | Dynamic | 255 | 11 | 6 | 11.2 | 8 | 858 |
| | | Duo | | | | | | |
| 20 | 4 | Front of | 252 | 12 | 1 | 12.6 | 8 | 572 |
| | | the Bus* | | | | | | |

From these additional measures, some observations suggested that there was a negative correlation between the passage reading time and number of correct answers given (R=−0.4) and a positive correlation between passage reading time and the number of going back to the passage (R=0.45). These may mean that as the students read the passages longer (harder passages to comprehend) their number of correct answers drops and they feel the need to go back to the passage more. There was a positive correlation between the session numbers and the passage reading time (R=0.42) and overall testing time (R=0.38) which may mean that students needed more time as they took the next tests during the day that may be related with a fatigue effect. There was a negative correlation between number of correct answers given and the question response time (R=−0.54) which may mean that students answer questions correctly in shorter time.

Averages in terms of students, sessions and passages can also be obtained. Averages over students are summarized in Table 5 below. It can be seen that student #20, read the passages the quickest, visited the passages the most times, answered the questions in shortest time and given the most number of correct answers with less number of tries as compared to the others. Subject #15 took the longest time to read the passages, visited the passages in intermediate levels, took the most time to answer the questions and tried several times to provide an answer, though had given the less number of correct answers on the average.

TABLE 5
Averaged values for each subject

| | | Average | | Average | Average | |
|---|---|---|---|---|---|---|
| Sub- | Passage | # of | Average # | Correct | Response | Overall |
| ject | Time (s) | Answers | of GoEssay | Time (s) | Answers | Time (s) |
| 10 | 175.75 | 12 | 3 | 6.5 | 14.98 | 478 |
| 15 | 216.25 | 16.25 | 5 | 6 | 18.6 | 538.5 |
| 20 | 168.75 | 10.75 | 9 | 7.5 | 14.94 | 517 |

If averages in terms of sessions (1 through 4) are carried out the detailed results of system 100 provide more correlations on certain fields. Table 6 summarizes the subject averaged measures of system 100 in terms of sessions. With this grouping, the correlation between the number of correct answers given and the average response time becomes R=−0.80.

TABLE 6
Subject averaged values for each session

| | | Average | Average | Average | Average | |
|---|---|---|---|---|---|---|
| Sub- | Passage | # of | # of | Correct | Response | Overall |
| ject | Time (s) | Answers | GoEssay | Answers | Time (s) | Time (s) |
| 1 | 198.33 | 14.33 | 1.00 | 6.00 | 17.30 | 420.33 |
| 1 | 91.33 | 13.33 | 0.33 | 7.00 | 16.73 | 387.00 |
| 3 | 181.33 | 12.00 | 2.67 | 7.67 | 14.27 | 669.33 |
| 4 | 276.67 | 12.33 | 1.67 | 6.00 | 16.39 | 568.00 |

If averages in terms of passages are carried out to eliminate the effects of difficulty levels of passages are carried out, the results become as given in Table 7. With this grouping, between the number of correct answers given and the average response time becomes R=−0.88.

TABLE 7
Subject averaged values for each passage

| | | | | | Average | Over- |
|---|---|---|---|---|---|---|
| | | Average | Average | Average | Re- | all |
| | Passage | # of | # of | Correct | sponse | Time |
| Passage | Time (s) | Answers | GoEssay | Answers | Time (s) | (s) |
| Liger's Tale | 124.33 | 11.33 | 1.00 | 7.00 | 13.37 | 310.67 |
| Dynamic Duo | 187.00 | 14.00 | 2.67 | 7.67 | 16.00 | 787.00 |
| Hatchet | 172.67 | 13.33 | 1.00 | 6.33 | 17.43 | 397.33 |
| Front of the Bus | 252.00 | 12.00 | 1.00 | 8.00 | 12.66 | 572.00 |
| Phantom | 269.50 | 14.00 | 1.00 | 4.50 | 20.50 | 538.50 |
| Tollbooth | | | | | | |

These preliminary analyses on the behavioral outcomes as measured by system 100 are carried out to provide examples on how system 100 can be used to obtain more detailed and elaborate evaluation of student performances on reading comprehension tests. Each individual student can be evaluated on certain measures within themselves over various testing time points or across each other at a given time point or over time in terms of improvement/decline. Additional analysis can also be carried out at various grade levels. All the detailed information that system 100 provides in terms of passage viewing, number of answers given, timings of answers and so forth provide previously unattainable information by the use of paper and pencil tests.

Brain-based measures from fNIRS were recorded in the following manner. Raw intensity measurements at 730 and 850 nm wavelengths are first filtered with a finite impulse response (FIR) filter to eliminate heart pulsation, respiration and high frequency noise signals. Then using the modified Beer-Lambert law, raw intensity measurements are converted into changes in Oxy-Hb and Deoxy-Hb relative to the 10 sec baseline period collected at the beginning of the measurement.

Using the timings of recordings by system 100, data epochs from the questions asked response given is extracted for each student, passage, channel, hemodynamic variables (Oxy- and Deoxy-Hb) and question. The epochs are baseline corrected (mean of pre epoch region is subtracted from the epoch) to eliminate the effects of pre-epoch activities from the epoch region itself for normalization. Then maximum amplitude of each epoch of each hemodynamic variable which is a common feature used in fNIRS studies is extracted. Since Oxy-Hb has been shown to correlate well with cognitive activity and produce comparable results to fMRI findings, in this study analysis was first focused on Oxy-Hb results.

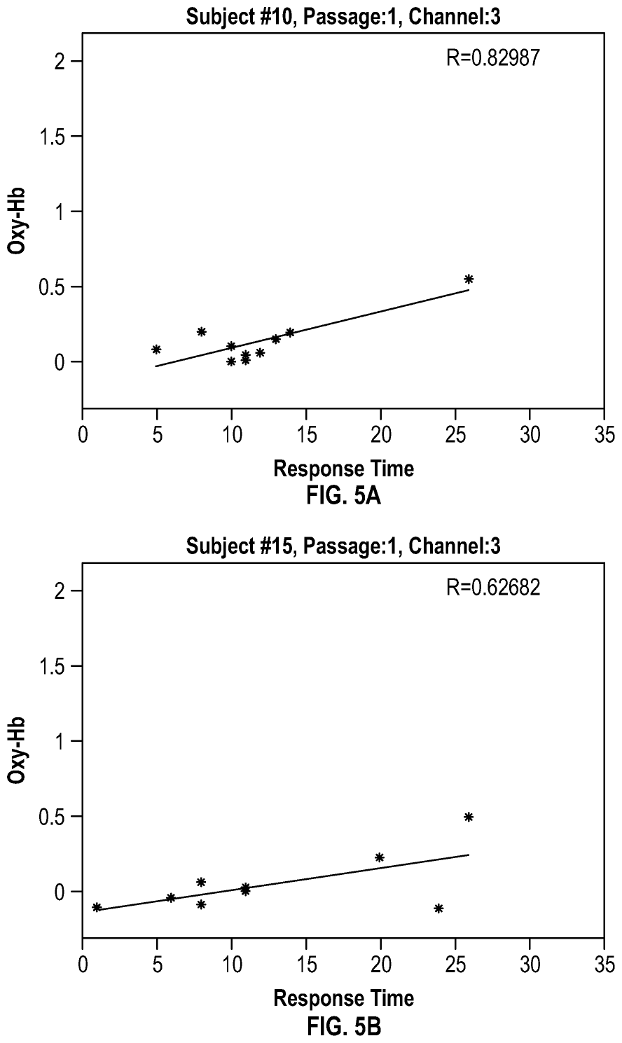

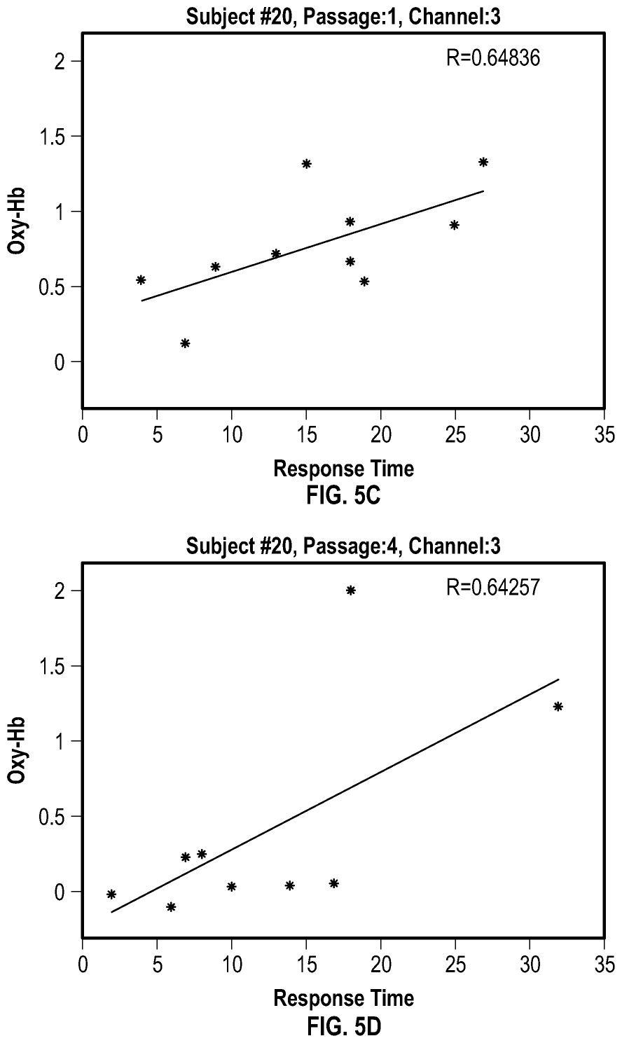

As an initial analysis the maximum Oxy-Hb values of each of the 10 question epochs are correlated with the corresponding behavioral response times for each individual subject and test where fNIRS measures were collected (as given in Table 2), separately. On channel 3 (middle frontal area on the right hemisphere, which corresponds to attentional domains as found out in previous fNIRS and fMRI studies), high correlation values were found, as summarized in Table 8 below. In FIGS. 5A-5D, scatter plot of fNIRS values on channel 3 vs response times for each fNIR recording session is given. These preliminary results indicate that there is a positive correlation between subject's response time and maximum Oxy-Hb values meaning that when subjects spend more time and effort in a question, the oxygenation in a certain area of the brain increases accordingly.

TABLE 8
Correlation values between Oxy-Hb and response times

| | Subject #10, | Subject #15, | Subject #20, | Subject #20, |
|---|---|---|---|---|
| | passage 1 | passage 1 | passage 1 | passage 4 |
| R | 0.829 | 0.626 | 0.648 | 0.642 |

Average values of maximum Oxy-Hb were calculated in all questions for each subject and passage where there is fNIRS recording. These values are summarized in Table 9 below.

TABLE 9
Correlation values between Oxy-Hb and response times

| | | | Pas- | Over- | | | | Re- |
|---|---|---|---|---|---|---|---|---|
| | | | sage | all | # of | | | sponse |
| Sub- | | | Time | Time | An- | # of | Correct | Time |
| ject | Passage | HbO2 | (s) | (s) | swers | GoEssay | Answers | (s) |
| 10 | Liger's | 0.141 | 123 | 259 | 11 | 0 | 7 | 12 |
| | Tale | | | | | | | |
| 15 | Liger's | 0.050 | 108 | 292 | 13 | 1 | 7 | 12.6 |
| | Tale | | | | | | | |
| 20 | Liger's | 0.785 | 142 | 381 | 10 | 2 | 7 | 15.5 |
| | Tale | | | | | | | |
| 20 | Front of | 0.423 | 252 | 444 | 11 | 1 | 8 | 12.7 |
| | the Bus | | | | | | | |

It was found that there were positive correlation between the Oxy-Hb values and overall testing time (R=0.67), number of times the passage has been viewed (R=0.79) and the average response time (R=0.89). These results mean that as it takes for certain subjects more time to complete the test and they need more revisiting the passage, they have to put more effort in it and hence their response times and the corresponding Oxy-Hb values increase.

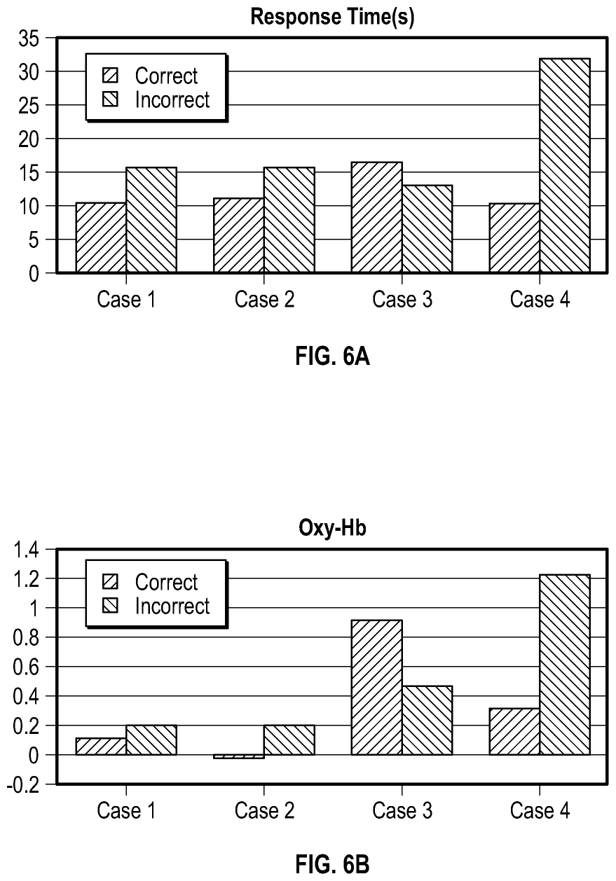

The correct and incorrect responses were separated and calculated the average maximum Oxy-Hb and response times for each subject and passage as summarized in Table 10 below. Similar information is also given in FIGS. 6A and 6B for better visual inspection.

TABLE 10
Correct vs incorrect answers Oxy-Hb and response time values

| | | Oxy-Hb | | # of Answers | | Response Time (s) | |
|---|---|---|---|---|---|---|---|
| Sub- | | | In- | | In- | | In- |
| ject | Passage | Correct | correct | Correct | correct | Correct | correct |
| 10 | Liger's | 0.115 | 0.200 | 7 | 3 | 10.429 | 15.667 |
| | Tale | | | | | | |
| 15 | Liger's | −0.017 | 0.207 | 7 | 3 | 11.286 | 15.667 |
| | Tale | | | | | | |
| 20 | Liger's | 0.918 | 0.474 | 7 | 3 | 16.571 | 13.000 |
| | Tale | | | | | | |
| 20 | Front of | 0.321 | 1.236 | 8 | 2 | 10.250 | 32.000 |
| Av- | the Bus | | | | | | |
| erage | | 0.334 | 0.529 | 7.25 | 2.75 | 12.134 | 19.083 |

All cases had more correct answers than incorrect ones. On the average, incorrect answers took more time to respond and more Oxy-Hb. Individually also, incorrect answers took in general more time to answer and more Oxy-Hb. Only in subject 20, passage “tiger's Tale” did it take less time for incorrect answers, but in this case, it corresponded to less Oxy-Hb in incorrect answers as compared to the correct ones.

This example only used readers of native English speaker within the same grade level and compared their behavioral results based on system 100 with their brain measures. Those skilled in the art, however, will recognize that system 100 can be used with individuals with specific learning disabilities in reading, individuals of different age and grade groups, individuals where English is a second language and compare their outcomes using system 100 within and across groups together with their brain measures.

It is expected that system 100 will be able to provide the following information that can be used to inform instruction. Such information can include:

1. How long it took the student to read the passage through to the first question.

2. How long it took the student to answer each question.

3. If the student referred back to the passage while answering a question.

4. If the student got the answer correct or incorrect.

5. Which answer the student chose and why it was the wrong answer (heuristic).

6. Total percentage of answers correct.

7. Types of wrong answers and how many of each.

8. A graph with the data, Lexile® level and score for the student for the school year.

9. How long the entire passage with questions took to read and answer.

10. A warning when a student has not shown progress for three sessions in a row.

11. A signal when student has read three passages at that grade Lexile® level with 75% or more accuracy—which is a signal for the teacher to move the student to the next level.

12. A class roster with student names highlighted in colors such as: green (on target); yellow (just below target); and red (well below target) for graded Lexile level.

13. Strategies for working with students depending on the type of wrong answers selected by the students.

14. Ability for student to read orally into a cloud based system to enable the teacher to hear reading fluency of the students.

15. Ability for an iPad to read passage to a student who may have difficulty decoding and teacher wants to check listening comprehension.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A method for developing a reading comprehension test for readers, the method comprising the steps of:
(a) providing at least one passage of text;
(b) providing a question based on the passage; and
(c) providing a plurality of potential answers, the potential answers having only a single correct answer and at least three incorrect answers, each answer having a different answer construct;
wherein a first of the incorrect answers uses brain frontal lobe activity and wherein a second of the incorrect answers does not use brain frontal lobe activity, wherein the answer construct for the second of the incorrect answers attracts readers who do at least one of the following:
(i) rely on prior knowledge; or
(ii) do not read or comprehend the passage.

2. The method according to claim 1, further comprising the steps of:
(d) providing a test subject wearing a functional near infrared device of the test subject's head;
(e) providing the passage, the question, and the plurality of potential answers to a test subject;
(f) requiring the test subject to read the passage and answer the question; and
(g) determining whether brain frontal lobe usage is measured by the functional near infrared spectroscopy device.

3. The method according to claim 2, further comprising the step of:
(h) repeating steps (d)-(g) for a plurality of test subjects.

4. The method according to claim 3, further comprising the step of:
(i) if more than a predetermined number of the plurality of test subjects did not use brain frontal lobe activity, discarding the question.

5. The method according to claim 3, further comprising the step of:
(i) if not more than a predetermined number of the plurality of test subjects did not use brain frontal lobe activity, using the question for subsequent testing.

6. The method according to claim 1, wherein step (c) further comprises attracting readers who do at least one of the following:
(iii) do not read a prompt in the question; or
(iv) cannot use context to determine a meaning of figurative text.

7. The method according to claim 1, wherein the single correct answer requires brain frontal lobe usage.

8. A method for developing a reading comprehension test for readers, the method comprising the steps of:
(a) providing at least one passage of text;
(b) providing a question based on the passage; and
(c) providing a plurality of potential answers, the potential answers having only a single correct answer and at least three incorrect answers, each answer having a different answer construct;
wherein a first of the incorrect answers uses brain frontal lobe activity and wherein a second of the incorrect answers uses less brain frontal lobe activity than the first of the incorrect answers.

9. The method according to claim 8, wherein the answer construct for the second of the incorrect answers relies on supportive, evidentiary facts not in the text.

10. The method according to claim 8, wherein the answer construct for the second of the incorrect answers provides a text-based literal fact.

11. The method according to claim 8, wherein the answer construct for the second of the incorrect answers provides a reasonable answer related to the question, but is not a text-based fact.

12. The method according to claim 8, wherein the answer construct for the second of the incorrect answers relies on common background knowledge not in the text.

13. The method according to claim 8, wherein the answer construct for the second of the incorrect answers provides a text-based fact related to a character in the text, but is not related to a point of view of the character.

14. The method according to claim 8, wherein the answer construct for the second of the incorrect answers provides evidence from the text, but does not support a character point of view.

15. The method according to claim 8, wherein the frontal lobe activity includes both left I and right frontal brain activity.

16. The method according to claim 8, wherein the answer construct for the second of the incorrect answers provides a reasonable literal meaning of a phrase in the text.

17. The method according to claim 8, wherein the answer construct for the second of the incorrect answers provides a reasonable answer to the question, but is not related to an overall structure of the text.

18. The method according to claim 8, further comprising the steps of:
(d) providing a test subject wearing a functional near infrared device of the test subject's head;
(e) providing the passage, the question, and the plurality of potential answers to a test subject;
(f) requiring the test subject to read the passage and answer the question; and
(g) determining whether brain frontal lobe usage is measured by the functional near infrared spectroscopy device.

19. A method for developing a reading comprehension test for readers, the method comprising the steps of:
(a) providing at least one passage of text;
(b) providing a question based on the passage; and
(c) providing a plurality of potential answers, the potential answers having only a single correct answer and at least three incorrect answers, each answer having a different answer construct, wherein each of the different answer constructs is directed to determining a different goal.

20. The method according to claim 19, wherein a test taker's selection of the single correct answer indicates left brain frontal lobe usage to select the single correct answer.
